(19) United States
(12) Patent Application Publication
(10) Pub. No.: US 2005/0191206 A1
(43) Pub. Date: Sep. 1, 2005

(54) Decontamination of biological spores with mild acid and moderate heat

(75) Inventors: Tony L. Buhr, King George, VA (US); Lindsay A. Sobota, Spotsylvania, VA (US); Amanda S. Schilling, Fredericksburg, VA (US); Bradford W. Gutting, Fredericksburg, VA (US); Ryan S. MacKie, Spotsylvania, VA (US); Andrew F. Slaterbeck, King George, VA (US); Alfredo N. Rayms-Keller, Fredericksburgh, VA (US)

Correspondence Address: NAVAL SURFACE WARFARE CENTER, DAHLGREN DIVISION;OFFICE OF COUNSEL, CODE XDC1, 17320 DAHLGREN ROAD, DAHLGREN, VA, 22448-5110, US

(73) Assignee: Government of the United States of America as represented by the Secretary of the Navy

(21) Appl. No.: 10/794,548

(22) Filed: Feb. 27, 2004

(51) Int. Cl. A61L 2/00

(52) U.S. Cl. 422/28; 422/12; 422/17; 422/307

(57) ABSTRACT

A decontaminating method for biological spore populations uses the application of an acidic environment to the biological spores with an additional step of moderately heating the biological spores in the acidic environment to decontaminate the spores.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefore.

### BACKGROUND OF THE INVENTION

1. Field of the Invention

The present invention provides a method for decontamination of biological spores.

2. Brief Description of the Related Art

Several methods and treatments for killing and decontaminating bacterial spores are known, which include treatment with concentrated levels of strong acids or high heat such as autoclaving (>100° C.). When used in decontamination processes, strong chemicals are environmentally toxic, hazardous to people and destructive to valuable items, such as electronics and paper products. High heat, such as that generated by autoclaving, presents logistical problems and is unrealistic for most decontamination applications.

There is a need in the art to provide an effective and mild decontamination methodology. The present invention addresses this and other needs.

### SUMMARY OF THE INVENTION

The present invention includes a method for decontaminating biological spores comprising the steps of applying an acidic environment to biological spores and moderately heating the biological spores in the acidic environment effective to decontaminate the biological spores. The method results in the decontamination of a spore population.

The use of mild, dilute acid and moderate heat for decontamination of bacterial spores provides an environmentally friendly methodology compared to using strong acid or high heat individually.

### BRIEF DESCRIPTION OF THE DRAWINGS

FIG. 1 is a graph plotting the number of live B. globigii spores at Time 0 (control), the number of live spores following 10 mM oxalic acid treatment for 30 minutes at 25° C., and then an additional 30-minute incubation in oxalic acid at 70° C.;

FIG. 2 is a graph plotting B. globigii spore germination and growth compared to vegetative bacterial growth at pH 5.0, pH 6.0, pH 7.0, pH 8.0 and pH 9.0;

FIG. 3 is a graph plotting spore population for oxalic acid treatment in the presence and absence of external heat, and in the presence and absence of acid neutralization;

FIG. 4 is a graph plotting spore population for oxalic acid treatment with no room temperature (20° C.-25° C.) pre-incubation step; and,

FIG. 5 is a graph plotting spore population for acetic acid treatment with no room temperature (25° C.) pre-incubation step.

### DETAILED DESCRIPTION OF THE PREFERRED EMBODIMENT

The present invention provides a method for decontaminating biological spores using the application of an acidic environment to spore populations under moderately heated conditions. The method of the present invention decontaminates the spores with minimal environmental risks. This allows effective decontamination of fragile objects without damage or destruction occasioned by decontamination under more severe conditions.

The present invention is applicable for decontamination of biological spores, particularly biological spores that comprise bacterial endospores. As used herein, the terms spores, biological spores, spore populations and similar terminology, refer to contaminant spores that create a hazard, threat, nuisance, etc. by their presence in an environment, on a surface, in food, etc. Typical spores decontaminated by the present invention include, for example, endospores, such as those belonging to the genus Bacillus, Clostridium, and the like. Representative endospore populations include Bacillus and Clostridium species. Some examples are Bacillus subtilis, Bacillus anthracis and Bacillus globigii.

Effectiveness of the methodology of the present invention occurs with increases of biological spore kills with the use of the acidic environment plus heat over non-use of such conditions. Preferably, an effective kill is variable, depending on the original number of spores within a contamination, such as a 90% effectiveness (kill) against a concentration of 10³ spores/ml, and more preferably an effectiveness of 90% against a concentration of 10⁸ spores/ml, with a most preferred decontamination of from about six or more logarithmic reductions of live spores. Most preferably, the decontamination reduces the spore concentration to a level that renders the once hazardous contaminated area or surface no longer hazardous. Effective biological spore decontamination of these spores rids a contaminated space or object of the immediate hazard occasioned by the spore presence. Spores are killed when they are rendered harmless, i.e., no longer hazardous, to a living organism, particularly a human. Depending on the circumstances, spore decontamination may be desirable against spores that affect other mammals, animals or plants. Decontamination applications non-exclusively include decontamination of endospore-forming bacteria in military, medical, industry, agriculture, and household domains, particularly in the event of accidental contamination or terrorist attack. Representative localities that might benefit from the decontamination regimen of the present invention for reduction of spore populations include hospitals, veterinary clinics, farms, dairies, meat processing facilities, hide processing facilities, ships, buildings, houses, automobiles, and other like contaminated surfaces and/or areas.

Application of the acidic environment includes any appropriate methodology for exposing the spores, such as by surrounding, encasing, inundating, engulfing, submerging, misting, or otherwise subjecting the spores to the acidic environment as to affect the spores thereby. Methodologies may include sprays, mists, liquids, solids and the like. Preferably application of the acidic environment comprises application of an acidic solution to the spores or to an environment to which the spores are introduced. Acidic environments include, for example, pH ranges of less than about 7.0, preferably from about 1.0 to less than about 7.0, more preferably from about 2.0 to about 6.0, and most preferably from about 3.0 to about 4.0. Acidic solutions may include carboxylic acids, organic acids, inorganic acids, and combinations thereof, including such acids as oxalic acid, acetic acid, phosphoric acid, hydrochloric acid, sulfuric acid, nitric acids, and the like. In solution the acids may be present in amounts that allow the convenient and non-hazardous application onto the spores. Such amounts may include, for example, from about 1 mM to about 1,000 mM, from about 2 mM to about 500 mM, from about 10 mM to about 100 mM, from about 20 mM to about 50 mM, and the like. When solutions are applied to a spore population, preferably enough solution is applied to completely immerse the spores in the solution. Application of the acidic environment may include application of an acidic solution in a moderately heated condition.

The moderately heated condition of the spores includes above-ambient temperatures that in combination with the acidic environment decontaminate the spore population. Representative temperatures of a moderately heated condition include, for example, non-destructive temperatures for a given object to be decontaminated, such as temperatures below the boiling point of an aqueous constituent of the object to be decontaminated or any constituent part of the acidic environment, e.g., from about 100° C. or less. Preferred temperatures of the moderately heated condition include from about 35° C. to about 100° C., more preferably from about 50° C. to about 100° C., still more preferably from about 65° C. to about 85° C., and most preferably from about 75° C. to about 80° C. Methods of heating may include imparting heat into the acidic environment/spore population including, for example, use of exothermic chemical reaction, use of external heat source, and the like.

Methodologies of the decontamination of the spores include the spores being subjected to the acidic environment, and simultaneously or sequentially, exposed to a heated condition. Heat may be applied prior to, during or after the application of the acid. Acidic and heated conditions may be varied by the type and amount of spore population, and by the object or environment to be decontaminated. In one preferred embodiment, the biological spores are collected from a contaminated area and placed in a container for application of dilute acid and heat, such as in the form of a heating element. Alternatively, the application of preheated dilute acid is used for decontamination on site.

The type and amount of acid useful for decontamination of a particular spore population may be determined by those skilled in the art of decontamination in light of the disclosure herein. Relevant factors useful in determining the most appropriate heated acidic conditions to be used include such items as the type of spore species, type of surface or area to be decontaminated, amount of contamination, environmental conditions of the cleanup, and other such criteria that are determinable by those skilled in the area of decontamination.

In one application of the present invention, spores may be harvested and/or trapped by vacuums, filters, glues, etc. to collect and concentrate the spores and placed into a container or similar retaining device. Within the container, the spores are exposed to appropriate acidic conditions, which may be present when the spores were placed in the container or added after placement of the spores, and heated with a heating element for an appropriate time period to kill and decontaminate the spores. Such decontamination devices may be small, inexpensive and readily transportable, having an acid resistant container, such as a glass-based, resilient plastic or stainless steel composition, and a heating component such as a heating element. In a second embodiment, an exothermic chemical reaction may be used in place of the heating element for imparting heat to the acidic environment to kill spores in contaminated areas. Numerous factors, determinable by artisans in the decontamination arts, affect the efficiency of the decontamination method of the present invention. Such factors include, for example, the selection of acid, acid concentration, uniformity of heat, variations in heat temperatures, time period of application, type of spore, etc., with the optimum decontamination conditions determinable by those skilled in the art through routine experimentation in light of the disclosure herein. Generally, selection of appropriate decontamination procedures includes a balance of acid strength, heat conditions, time constraints (such as operational military timetables), environmental sensitivities to acidic and/or elevated heated conditions, and toxicity for a given spore hazard.

The present invention may include optional components such as catalysts, surfactants, sodium carbonate, sodium hydroxide, water, enzymes, and the like. Enzymes are frequently useful for decontaminating vegetative bacteria and may decontaminate some spores when applied with the mild acid solution. Preferred amounts of enzymes include approximately 1 mg/ml with activity of from about 10 to about 20 units per mg.

Advantageously the present invention provides low toxicity, low cost, high availability of acids and reduced logistical problems for decontamination. Selection of toxicity, kill ratios for a given spore population, and heat conditions may be varied to treat specific contaminated environments or surfaces. Within working ranges of the present invention, such as typical amounts of 10 mM or 100 mM, acids are dilute, inexpensive and readily available. Logistical concerns are mitigated because acids can be transported in small volumes of concentrated amounts and diluted prior to use. These dilute acids of the present invention have minimal toxicities for decontamination of a given hazard, and are much more environmentally friendly than the same acids in more concentrated amounts.

Experiments were performed to show effective spore kill ratios using a combination of mild heat and mild, dilute acid. As detailed in the examples, below, Example 1A presents an analysis of a germination assay, Example 1B describes another germination assay (LB agar), showing the affect of the pH variable on spore germination and bacterial growth, and Example 1C presents neutralization experiments in liquid assays.

### EXAMPLE 1A

### Spore Germination Experiments in Liquid Assays

Germination includes developmental processes such as all physiological, morphological, and biochemical changes that occur as a dormant spore transitions into a vegetative bacterium. One characteristic of germination is loss of heat resistance. Dormant spores are heat resistant, such as at elevated temperatures of 70° C. Germinating or germinated spores, and vegetative cells are very heat susceptible, and die at elevated temperatures.

Germination experiments were conducted in liquid solutions, using the following steps. All experiments were performed in triplicate.

B. globigii spores were suspended in a putative germinant solution on ice. From the suspended spores an aliquot of spores was removed, which was then serially diluted and plated on LB agar plates. This gave a live spore titer or colony-forming units (CFUs) at Time=0 minutes. The samples (spores in germinant solution) were incubated at room temperature (˜20° C.) for 30 minutes. An aliquot of incubated spores was then removed and serially diluted and plated on LB agar plates. This gave a live spore concentration at Time=30 minutes. After removing the aliquot at the 30-minute time point, the remainder of the undiluted samples were then incubated in a 70° C. water bath for 30 minutes. The samples were then serially diluted and plated on to LB agar plates to determine the titer of live spores after heat.

The 30-minute, room temperature incubation allowed spores to germinate. Spores that germinated during the room temperature incubation should have been killed during the subsequent 70° C. incubation, and spores that remained dormant should have remained alive during the 70° C. incubation. One limitation, however, of the quantitative assay was the inability to distinguish between germinated spores and spores that are damaged, since both germinated and damaged spores can be sensitive to 70° C. heat.

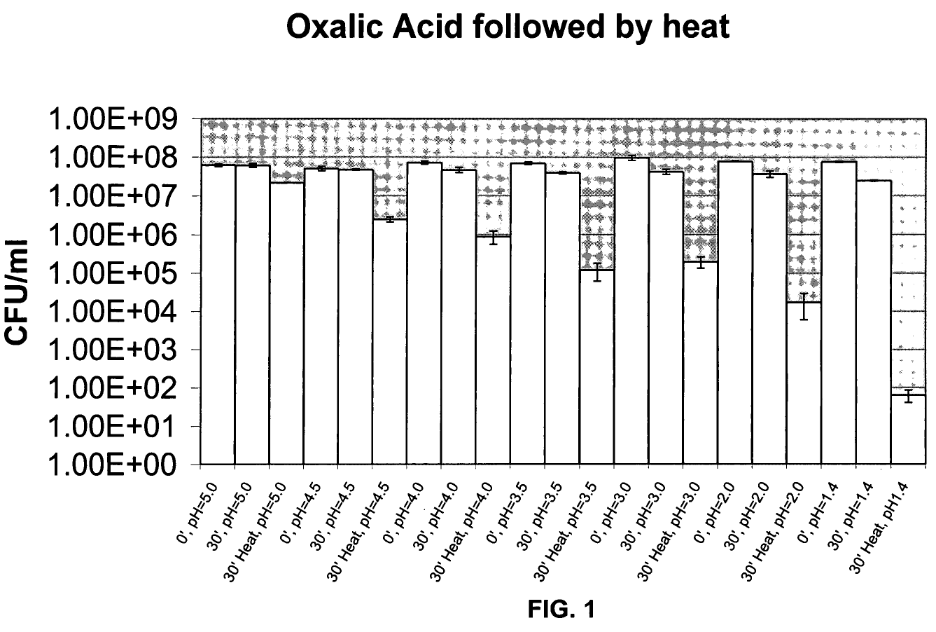

The preliminary germination experiment was performed using oxalic acid as a putative germinant. A final concentration of 10 mM oxalic acid was tested over a pH range between pH 1.4 and pH 5.0. As seen in the results shown in FIG. 1, the 10 mM oxalic acid worked best as the pH was lowered from pH 5.0 to pH 1.4. As the conditions were highly acidic pH levels, spores may have been damaged during the acid treatment and then killed by the heat with the ability to distinguish between germination and spore damage unavailable.

### EXAMPLE 1B

### Spore Germination Experiments on LB Agar, pH Variable

B. globigii spore germination and bacterial growth were examined on LB agar plates, which allowed testing of variable pH conditions. The number of CFU after plating spores was a measure of both spore germination and then bacterial growth. The number of CFU after plating vegetative bacteria was only a measure of bacterial growth. Custom-made LB agar plates were poured at pH 5.0, pH 6.0, pH 7.0, pH 8.0, and pH 9.0. LB agar plates below a pH of 5.0 were not available.

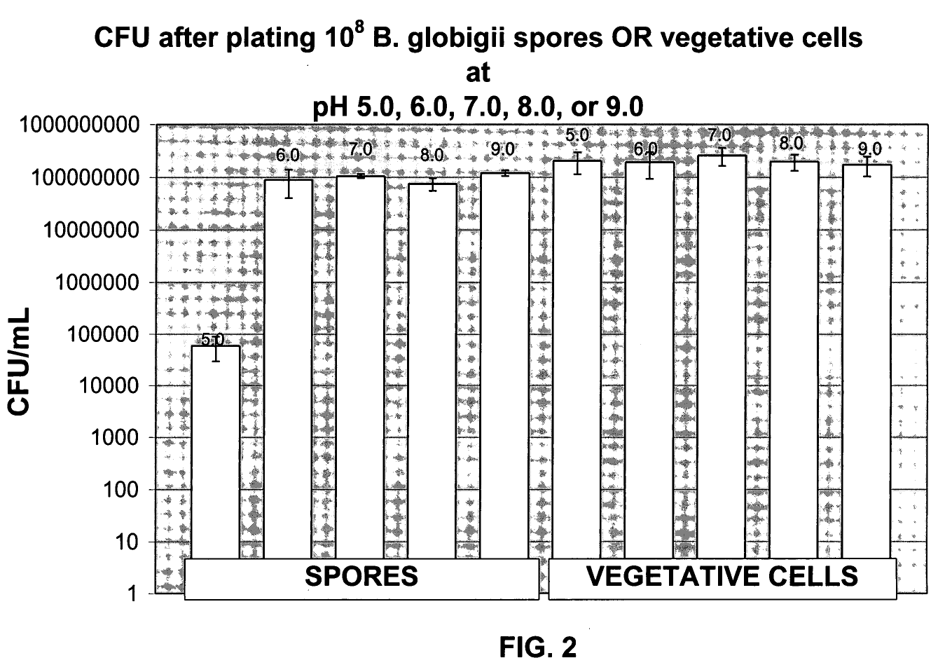

An approximately equal number of dormant B. globigii spores or metabolically active, log-phase vegetative bacteria were plated directly onto pH-adjusted LB agar plates. As shown in FIG. 2 there were fewer CFU after plating dormant spores at pH 5.0 than pH 6.0, pH 7.0, pH 8.0 or pH 9.0, with the pH 5.0 having greater than 3-log reduction (99.9%) after plating spores than at pH 6.0-9.0. It was also noted that the number of CFU after plating log-phase bacteria on LB agar was nearly the same at all pH's between pH 5.0 and pH 9.0.

These data showed that vegetative B. globigii grew well between pH 5.0 and pH 9.0, but spore germination was seriously hindered at pH 5.0. The dramatic decrease in germination that was observed as the pH was lowered from pH 6.0 to pH 5.0 also suggested that minimal germination occurs at pH<5.0. These data further suggest that the heat kill shown in FIG. 1 was not due to spore germination because most of the tests for FIG. 1 were performed at pH<5.0.

### EXAMPLE 1C

### Neutralization Experiments

The data from Example 1A and 1B indicated incubation of spores in increasingly acidic environments causes spore damage, and the damaged spores could be killed by heat. Investigation of the combination of acid plus heat to kill the spores was conducted. The experiment was designed to neutralize the acid after the room temperature incubation and before the heat step. Samples were neutralized with EPPS buffer (0.1M final concentration) to bring the pH between pH 8.0-8.7 for all samples. The neutralization experiments were conducted as follows: B. globigii spores were suspended in a acid test solution on ice. From the suspended spores an aliquot of spores was removed, which was then serially diluted and plated on LB agar plates. This gave a live spore titer or colony-forming units (CFUs) at Time=0 minutes. The samples (spores in test solution) were incubated at room temperature (˜20° C.) for 30 minutes. EPPS buffer, pH8.7, was added to the spores to neutralize the acid and give 100 mM final concentration of buffer. An aliquot of incubated spores was then removed and serially diluted and plated on LB agar plates. This gave a live spore concentration at Time=30 minutes. After removing the aliquot at the 30-minute time point, the remainder of the undiluted test samples was then incubated in a 70° C. water bath for 30 minutes. The samples were then serially diluted and plated on to LB agar plates to determine the titer of live spores after heat.

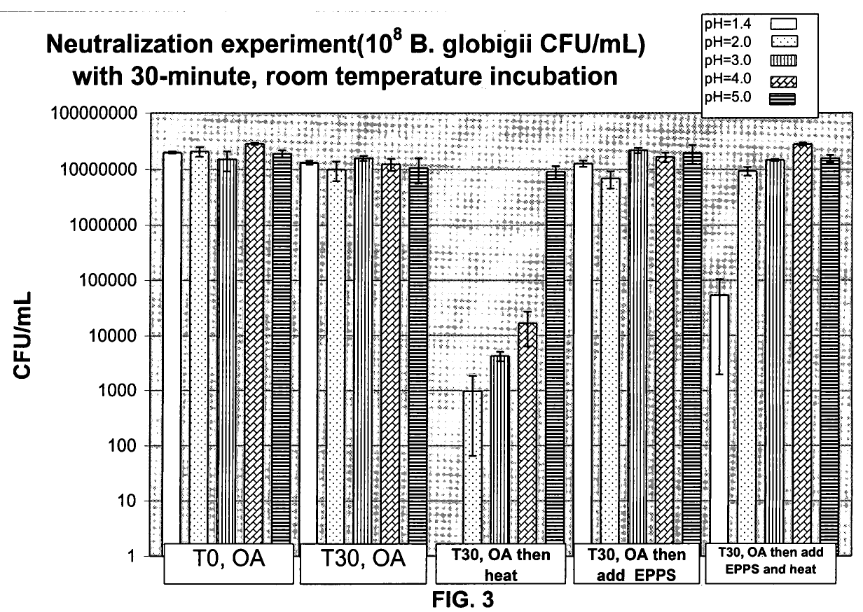

FIG. 3 shows the results of the neutralization experiments. There was little reduction in the number of CFU between Time 0 and Time 30 minutes for all pH's, indicating minimal spore death during this time period. However, acid plus heat samples that were not neutralized showed a decreasing number of live spores as pH decreased. Neutralization of the same samples prior to heat kill greatly reduced or eliminated the heat kill. This indicated the efficacy of the combination of the acid and heat for decontamination. One exception was that pH 1.4 still yielded a high level of heat kill even after neutralization. However, this was still not nearly the reduction seen without neutralization. These results indicated that a low enough pH damaged spores enough to render them heat susceptible, but the combination of acid plus heat has much higher efficacy than acid alone.

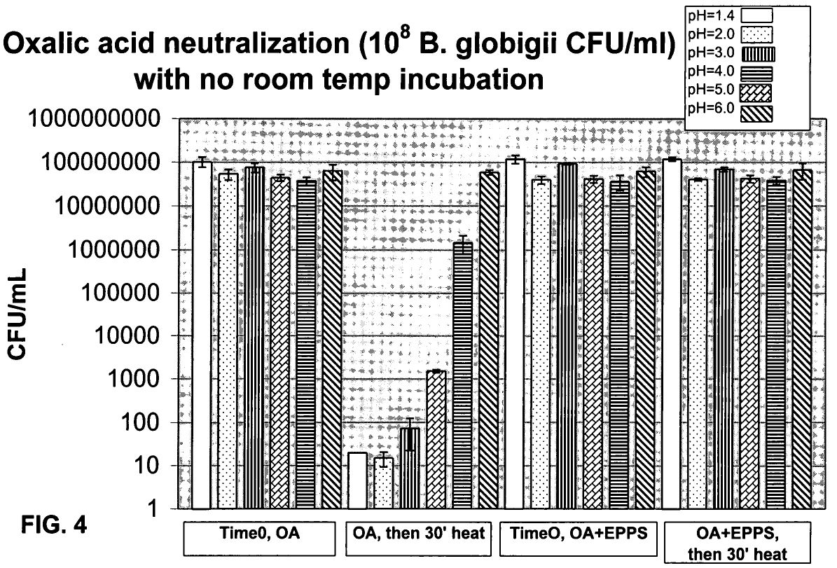

Decontamination regimes were also conducted when spores were not pre-incubated in acidic solutions prior to incubation at elevated heat. Experiments were conducted where the 30-minute, room temperature incubation period prior to heat treatment was eliminated. As seen in FIG. 4, the 30-minute room temperature incubation period was unnecessary. The combination of dilute acid and moderate heat was sufficient to kill spores without using a pre-incubation in dilute acid. The elimination of pre-incubation procedures provides a more time efficient decontamination process.

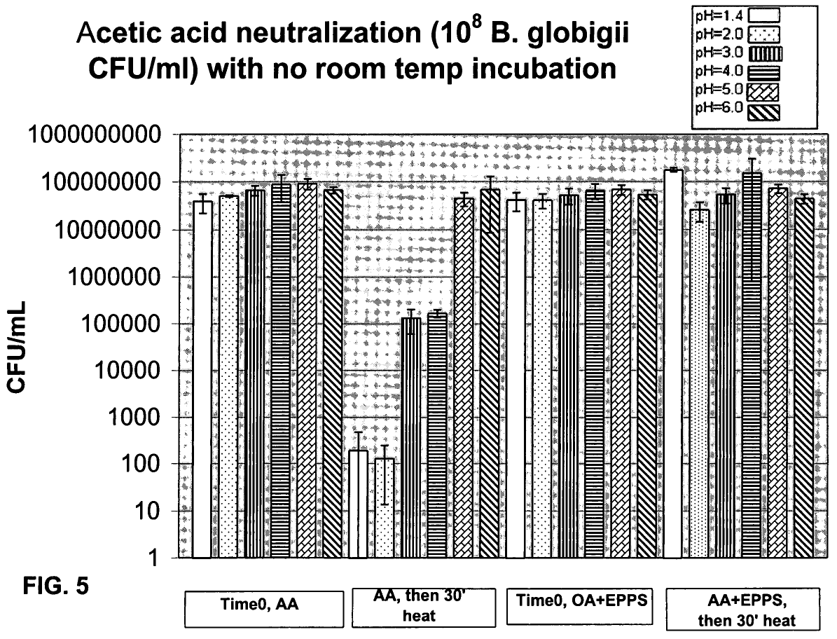

Alternative acids were investigated, with 10 mM oxalic acid replaced with 10 mM acetic acid. As shown in FIG. 5, the 10 mM acetic acid plus heat generated a similar pattern of spore decontamination as 10 the mM oxalic acid plus heat.

### EXAMPLE 2

### Prophetic

Air contaminated with Bacillus anthracis in an enclosed area (building, vehicles, planes, or other enclosed space) is purified using the present invention. As the Bacillus anthracis spores enter the intake duct in the building, the spores are removed by blowing the contaminated air up through a waterfall of dilute acid, which captures the spores thereby removing the spores from the air. The dilute acid/spore solution is then collected and circulated to a container with a heating element at approximately 70° C. for at least 30 minutes to kill the trapped spores. The dilute acid solution can then be re-circulated through the air-scrubbing chamber and re-used.

### EXAMPLE 3

### Prophetic

A hospital room becomes contaminated with bacterial spores from the presence of an anthrax victim. A dilute acidic solution is sprayed through a hot washer, which heats the mild acid to a temperature of at least 70° C., onto the walls, floor and ceiling of the hospital room. The mild acid is neutralized as it cools, and can be fully neutralized with a subsequent spraying of excess water or a mild buffer.

### EXAMPLE 3A

### Prophetic

The process of Example 3 is followed, with the exception that the contaminated space is a large concrete areas used for mass casualty treatment.

### EXAMPLE 3B

### Prophetic

The process of Example 3A is followed, with the contaminated space being a shower area.

### EXAMPLE 3C

### Prophetic

The process of Example 3 is followed, with the exception that the contaminated space is an industrial processing site.

### EXAMPLE 3D

### Prophetic

The process of Example 3 is followed, with the exception that the contaminated space is a processing area for the food industry.

### EXAMPLE 3E

### Prophetic

The process of Example 3 is followed, with the exception that the contaminated space is an agricultural buildings such as poultry buildings.

### EXAMPLE 4

### Prophetic

A kitchen surface is wiped down with a rag or cloth that is saturated with a mild acid solution. The applicator (rag or cloth) can then be collected and removed, and then heated in any type of heating device to kill the spores.

### EXAMPLE 5

### Prophetic

Human skin is wiped down with a rag or cloth that is saturated with a mild acid solution. The applicator (rag or cloth) can then be collected and removed, and then heated in any type of heating device to kill the spores.

### EXAMPLE 6

### Prophetic

Acid is added to water to an appropriate concentration. For example, citric acid (powder or liquid) is added to water to a final concentration of 10-100 mM and adjusted to pH=2. The water is then heated in any heating device to 70° C. for 30 minutes. The water is then cooled. The water can either be used directly or it can be pH neutralized with a base such as calcium hydroxide or sodium hydroxide (powder or liquid). The purified water can then be used for most applications including drinking, showering and washing clothes.

The foregoing summary, description, and examples of the present invention are not intended to be limiting, but are only exemplary of the inventive features which are defined in the claims.

1. A method for decontamination of a contaminated space from the presence of live biological spores, comprising the steps of:
applying an acidic environment to biological spores; and,
moderately heating the biological spores in the acidic environment effective to decontaminate the biological spores.

2. The method of claim 1, wherein the step of applying the acidic environment comprises application of an acidic solution.

3. The method of claim 2, wherein the step of applying the acidic environment comprises subjecting the spores to an acidic environment, and simultaneously or sequentially exposing the spores to a moderately heated condition.

4. The method of claim 1, wherein the step of applying the acidic environment comprises a pH of from about 1.0 to less than about 7.0.

5. The method of claim 4, wherein the step of applying the acidic environment comprises a pH of from about 2.0 to about 6.0.

6. The method of claim 5, wherein the step of applying the acidic environment comprises a pH of from about 3.0 to about 4.0.

7. The method of claim 2, wherein the acidic solution is selected from the group consisting of oxalic acid, acetic acid, phosphoric acid, hydrochloric acid, sulfuric acid, carboxylic acids, organic acids, inorganic acids and combinations thereof.

8. The method of claim 7, wherein the acidic environment comprises oxalic acid.

9. The method of claim 2, wherein the acidic solution is present in an amount of from about 1 mM to about 1,000 mM.

10. The method of claim 9, wherein the acidic solution is present in an amount of from about 2 mM to about 500 mM.

11. The method of claim 10, wherein the acidic solution is present in an amount of from about 10 mM to about 100 mM.

12. The method of claim 11, wherein the acidic solution is present in an amount of from about 20 mM to about 50 mM.

13. The method of claim 1, wherein the step of moderately heating the biological spores comprises a temperature of from about 50° C. to about 100° C.

14. The method of claim 13, wherein the step of moderately heating the biological spores comprises a temperature of from about 65° C. to about 85° C.

15. The method of claim 1, wherein the step of moderately heating the biological spores comprises an exothermic chemical reaction.

16. The method of claim 1, wherein the step of moderately heating the biological spores comprises an external heat source.

17. The method of claim 1, wherein the biological spores comprises endospores.

18. The method of claim 1, wherein the biological spores comprises Bacillus endospores.

19. The decontaminated space product produced by the method of claim 1, wherein the contaminating spores within the space have been rendered harmless.

20. The decontaminated space product produced by the method of claim 17, wherein the contaminating spores within the space have been rendered harmless.
